(19) United States
(12) Patent Application Publication
(10) Pub. No.: US 2009/0088706 A1
(43) Pub. Date: Apr. 2, 2009

(54) HEMORRHOID PAD AND METHOD FOR MAKING THE SAME

(75) Inventors: Jung-Chi Tai, Tainan Hsien (TW); Chun-Meng Huang, Tainan Hsien (TW); Ho-Hsi Yang, Tainan Hsien (TW); Chien-Chung Su, Tainan Hsien (TW)

Correspondence Address: BUTZEL LONG;IP DOCKETING DEPT, 350 SOUTH MAIN STREET, SUITE 300, ANN ARBOR, MI, 48104, US

(73) Assignee: KANG NA HSIUNG ENTERPRISE CO., LTD. Jung-Chi Tai, President, Tainan Hsien (TW)

(21) Appl. No.: 12/062,567

(22) Filed: Apr. 4, 2008

(30) Foreign Application Priority Data
Sep. 27, 2007 (TW) 096135947

(51) Int. Cl. A61F 13/00; B26D 7/34; A61L 15/00

(52) U.S. Cl. 604/304; 83/14; 427/2.31

(57) ABSTRACT

A hemorrhoid pad includes a folded laminated body having a folded end and two opposite folded halves diverging from the folded end. Each of the folded halves of the folded laminated body has inner and outer surfaces. The inner surfaces of the folded halves have a release agent coated thereon and face toward each other.

### CROSS-REFERENCE TO RELATED APPLICATION

This application claims priority of Taiwanese application no. 096135947, filed on Sep. 27, 2007.

### BACKGROUND OF THE INVENTION

1. Field of the Invention

This invention relates to a hemorrhoid pad, more particularly to a hemorrhoid pad having a folded end.

2. Description of the Related Art

A conventional hemorrhoid pad usually includes an absorbing layer and an adhesive formed thereon. The hemorrhoid pad is attached to the user's undergarment through the adhesive before use, which has a drawback in aligning the hemorrhoid pad with the infected area and in providing a suitable contact therebetween.

### SUMMARY OF THE INVENTION

Therefore, the object of the present invention is to provide a hemorrhoid pad that can overcome the aforesaid drawback associated with the prior art.

Another object of this invention is to provide a method for making the hemorrhoid pad.

According to the present invention, the hemorrhoid pad comprises a folded laminated body having a folded end and two opposite folded halves diverging from the folded end. Each of the folded halves of the folded laminated body has inner and outer surfaces. The inner surfaces of the folded halves have a release agent coated thereon and face toward each other.

According to another aspect of this invention, there is provided a method for making a hemorrhoid pad. The method comprises: (a) providing a sheet body having a surface coated with a release agent thereon; (b) folding the sheet body in such a manner that the surface coated with the release agent is formed into two surface halves facing toward each other; and (c) cutting the folded sheet body so as to obtain the hemorrhoid pad that is in a folded state.

### BRIEF DESCRIPTION OF THE DRAWINGS

Other features and advantages of the present invention will become apparent in the following detailed description of the preferred embodiment of this invention, with reference to the accompanying drawings, in which:

FIG. 1 is a perspective view of the preferred embodiment of a hemorrhoid pad according to this invention;

FIG. 2 is a perspective view to illustrate how the hemorrhoid pad is stored in a package;

FIGS. 3 to 5 are schematic views to illustrate consecutive steps of the preferred embodiment of a method for making a hemorrhoid pad; and

FIG. 6 is a schematic view to illustrate how the hemorrhoid pad is placed into the posterior rugae of the user.

### DETAILED DESCRIPTION OF THE PREFERRED EMBODIMENT

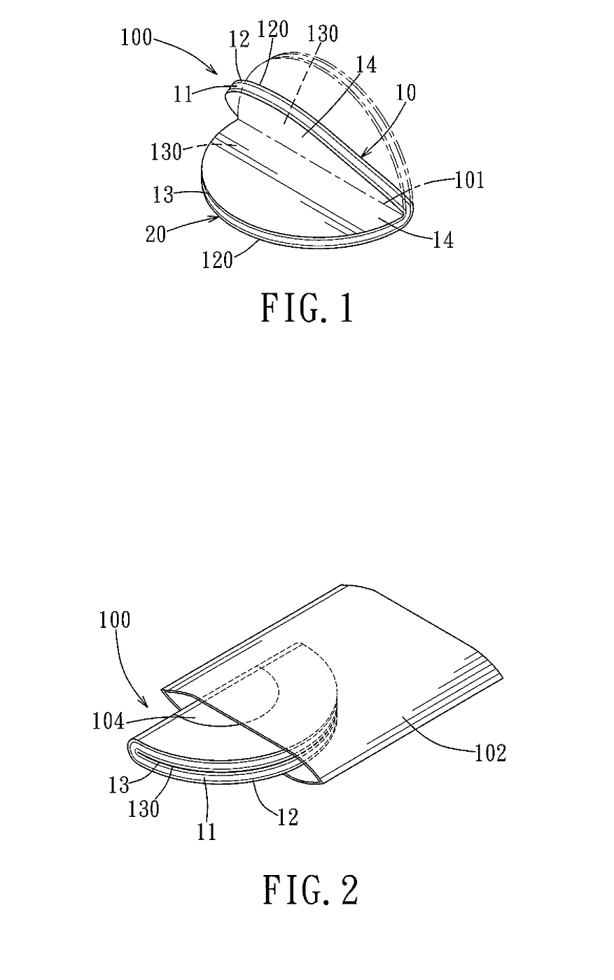

FIG. 1 illustrates the preferred embodiment of a hemorrhoid pad according to this invention.

The hemorrhoid pad includes a folded laminated body 100 having a folded end 101 and two opposite folded halves 10, 20 diverging from the folded end 101 to form an acute angle therebetween. Each of the folded halves 10, 20 of the folded laminated body 100 has inner and outer surfaces 130, 120. The inner surfaces 130 of the folded halves 10, 20 have a release agent 14 coated thereon, thereby forming a smooth surface, and face toward each other.

Preferably, the release agent 14 is coated on the inner surfaces 130 of the folded halves 10, 20 of the folded laminated body 100 by spraying or coating the release agent 14, or by attaching a release film.

In this embodiment, the folded laminated body 100 includes an absorbing layer 11 capable of absorbing liquid, and upper and lower layers 12, 13 sandwiching the absorbing layer 11 therebetween. The upper and lower layers 12, 13 define the outer and inner surfaces 120, 130 of the folded halves 10, 20 of the folded laminated body 100, respectively.

Preferably, the lower layer 13 is made from a material selected from the group consisting of polyethylene, polypropylene, polyethylene terephthalate, and combinations thereof.

Preferably, the lower layer 13 is made from one of a non-woven cloth and a paper having a waterproof film thereon.

Preferably, the lower layer 13 is made from a waterproof material.

Preferably, the outer surfaces 120 of the folded halves 10, 20 have an ointment 104 coated thereon (see FIG. 2).

Preferably, the folded halves 10, 20 are substantially the same in size, and are formed into a V-shaped configuration.

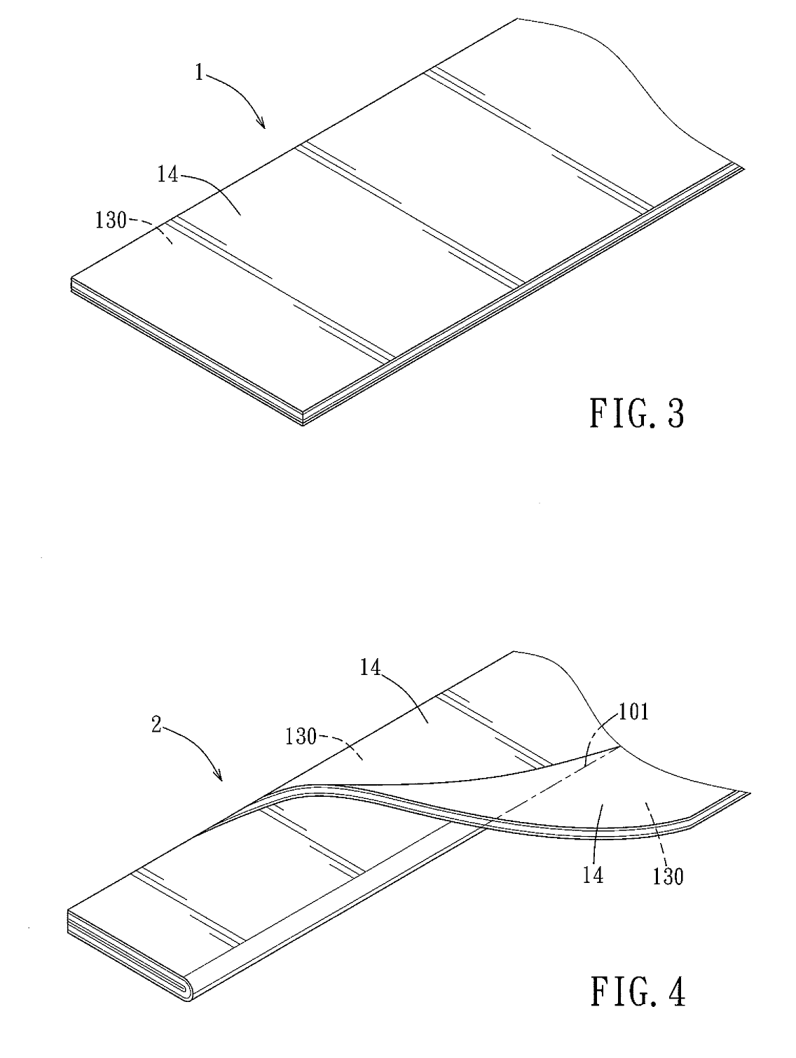

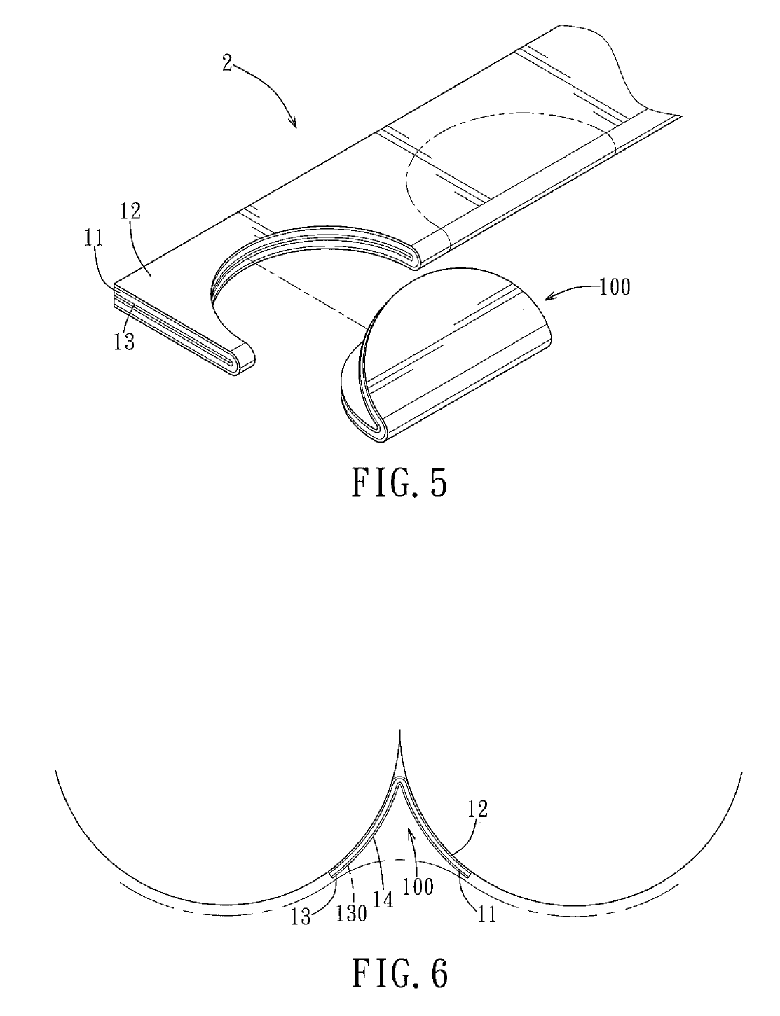

A method for making the hemorrhoid pad according to this invention is described as follows. Referring to FIGS. 3 to 5, the method includes the steps of: (a) providing a sheet body 1 having a surface, which forms the inner surfaces 130 of the folded halves 10, 20 of the folded laminated body 100, coated with a release agent 14 thereon; (b) folding the sheet body 1 in such a manner that the surface coated with the release agent 14 is formed into two surface halves facing toward each other; and (c) cutting the folded sheet body 2 so as to obtain the hemorrhoid pad, i.e. the folded laminated body 100, that is in a folded state.

Preferably, the folded laminated body 100 is made from an elastic material so that the folded halves 10, 20 can be further compressed into a thin stack and so that the folded laminated body 100 can be stored in a thin package 102 (see FIG. 2) prior to use. When the folded laminated body 100 is removed from the thin package 102, it can be automatically restored to its V-shaped configuration. Since the folded laminated body 100 is formed with the folded end 101, it can be easily and precisely placed into the posterior rugae (see FIG. 6), thereby eliminating the aforesaid drawback associated with the prior art.

With the invention thus explained, it is apparent that various modifications and variations can be made without departing from the spirit of the present invention. It is therefore intended that the invention be limited only as recited in the appended claims.

What is claimed is:

1. A hemorrhoid pad comprising:
a folded laminated body having a folded end and two opposite folded halves diverging from said folded end, each of said folded halves of said folded laminated body having inner and outer surfaces, said inner surfaces of said folded halves having a release agent coated thereon and facing toward each other.

2. The hemorrhoid pad of claim 1, wherein said folded laminated body includes an absorbing layer, and upper and lower layers sandwiching said absorbing layer therebetween, said upper and lower layers defining said outer and inner surfaces of said folded laminated body, respectively.

3. The hemorrhoid pad of claim 2, wherein said lower layer is made from a material selected from the group consisting of polyethylene, polypropylene, polyethylene terephthalate, and combinations thereof.

4. The hemorrhoid pad of claim 2, wherein said lower layer is made from one of a non-woven cloth and a paper having a waterproof film thereon.

5. The hemorrhoid pad of claim 2, wherein said lower layer is made from a waterproof material.

6. The hemorrhoid pad of claim 4, wherein said outer surfaces of said folded halves have an ointment coated thereon.

7. The hemorrhoid pad of claim 2, wherein said folded halves are substantially the same in size.

8. A method for making a hemorrhoid pad comprising:
(a) providing a sheet body having a surface coated with a release agent thereon;
(b) folding the sheet body in such a manner that the surface coated with the release agent is formed into two surface halves facing toward each other; and
(c) cutting the folded sheet body so as to obtain the hemorrhoid pad that is in a folded state.

9. The method of claim 8, wherein the release agent is coated on the surface of the sheet body by one of spraying and coating techniques.

10. The method of claim 8, wherein the release agent is coated on the surface of the sheet body by attaching a release film thereto.
